SEQUENCE LISTING (2 sequences)

SEQ ID NO: 1 - protein, 137 aa, Homo sapiens
MDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 2 - protein, 137 aa, Homo sapiens
  MUTAGEN (12)..(1)
MDIIMRPSCAPAWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

(19) United States
(12) Patent Application Publication
(10) Pub. No.: US 2013/0244954 A1
(43) Pub. Date: Sep. 19, 2013

(54) USE OF REG4 AND PHARMACEUTICAL COMPOSITION THEREOF

(71) Applicants: Wei Han, Shanghai (CN); Yan Yu, Shanghai (CN); Guoyong Hu, Shanghai (CN)

(72) Inventors: Wei Han, Shanghai (CN); Yan Yu, Shanghai (CN); Guoyong Hu, Shanghai (CN)

(73) Assignee: SHANGHAI REGENOME BIOMEDICAL TECHNOLOGY CO., LTD., Shanghai (CN)

(21) Appl. No.: 13/816,484

(22) PCT Filed: Aug. 8, 2011
(86) PCT No.: PCT/CN2011/001299

§ 371 Date: Apr. 16, 2013

(30) Foreign Application Priority Data
Aug. 13, 2010 (CN) 201010253097.9

(51) Int. Cl. A61K 38/17

(52) U.S. Cl. CPC A61K 38/17
USPC 514/21.2; 435/24; 435/29; 530/350

(57) ABSTRACT

The invention is in the field of biotechnology. Specifically it describes the application and pharmaceutical composition of Reg4. The invention describes the applications of the protein as following in (a) or (b) in preparing drugs for treating acute pancreatitis: (a) the protein whose amino acid sequence is shown as SEQ ID NO. 1, or bioactive fragments, or analogs; (b) the proteins whose amino acid sequence have at least 70% homology comparing with amino acid sequence described in (a) and have activity for treating acute pancreatitis. The protein described in this invention treats acute pancreatitis effectively and may provide new therapy for treating acute pancreatitis clinically.

### FIELD OF THE INVENTION

The present invention relates to the field of biotechnology, specifically the use of Reg4 and its pharmaceutical compositions.

### BACKGROUND OF THE INVENTION

Acute pancreatitis (AP) is a common clinical disease. In western countries, the annual incidence of AP is 30/100000, and approximately 15-20% of patients suffer from severe acute pancreatitis (SAP). Although the treatment of SAP has made important progress in recent years, the mortality rate is still about 20% [Pandol, S. J., et al., Gastroenterology, 2007. 133(3): p. 1056 e1-1056 e25.]. In addition, the majority of survival SAP patients are left with different degrees of pancreatic insufficiency in internal and external secretory function, and some of them develop to chronic pancreatitis. Histologically their pancreas shows acinar atrophy and interstitial fibrosis. Therefore, developing a new drug which treats AP, effectively reduces the mortality of SAP, and promotes the recovery of pancreatic function, is an urgent clinical need.

At present, the pathogenesis of AP has several hypothesis, including “trypsin self-digestion”, “over activated leukocyte”, “cascading waterfall effect of inflammatory cytokines”, “blood circulation disorder of pancreas”, “bacterial translocation of intestine, endotoxin blood disease and infection secondary heat”, “apoptosis”, and so on, which eventually lead to a systemic inflammatory response syndrome (SIRS) and even multiple organ failure (MOF). AP is a common disease, especially SAP, which lacks the ideal medication [Frossard, J. L., M. L. Steer, and C. M. Pastor, Lancet, 2008. 371(9607): p. 143-52]. In recent years, although the treatment of SAP has made a series of important progress, most patients have different degrees of pancreatic insufficiency in internal and external secretory function due to insufficient regeneration of pancreatic cells. Minority of the patients even develop chronic pancreatitis [Yasuda, T., et al., J Hepatobiliary Pancreat Surg, 2008. 15(4): p. 397-402].

Regenerating gene (Reg) family belongs to the calcium-dependent phytohemagglutinin superfamily [Lasserre, C., et al., Eur J Biochem, 1994. 224(1): p. 29-38.; Chakraborty, C., et al., Endocrinology, 1995. 136(5): p. 1843-9; Hartupee, J. C., et al., Biochim Biophys Acta, 2001. 1518(3): p. 287-93]. The Reg family is divided into four subtypes: Reg 1, 2, 3 and 4, wherein Reg2 is not expressed in human. Reg4 is a new Reg family member which was screened out by Hartupee Hartupee, J. C., et al., family: Reg IV. Biochim Biophys Acta, 2001. 1518(3): p. 287-93] from a library of inflammatory bowel disease. The expression of Reg4 shows tissue specificity with high expression in gastrointestinal tract and low in normal pancreatic tissue. Because the receptor of Reg4 has not been found, the biological function of Reg4 and its signaling pathway are poorly understood at present. The study of Takayama showed that REG4 is a specific marker of pancreatic cancer. The pancreatic cancer patients and healthy control subjects were discriminated by testing the serum level of REG4 [Takayama R, Nakagawa H, Sawaki A et al J Gastroenterol. 2010; 45(1):52-9].

### SUMMARY OF THE INVENTION

The present invention is to solve the technical problems of current treatment lacking effectiveness on acute pancreatitis.

To this end, the present invention discloses a use of the following protein (a) or (b) for preparing a medicament for the treatment of acute pancreatitis:

(a) a protein having an amino acid sequence as shown in SEQ ID NO. 1; (b) a protein which has a sequence having at least 70% homology with the amino acid sequence in the (a) and has the effect of treating acute pancreatitis.

In one embodiment, the acute pancreatitis is severe acute pancreatitis.

In one embodiment, the protein (b) is a protein having an amino acid sequence as shown in SEQ ID NO. 2.

In another aspect, the present invention discloses a pharmaceutical composition, which is composed by the following weight percentages of components:

The above proteins as active ingredients are composed from 1% to 99%, and the remaining are pharmaceutically acceptable carriers or excipients.

In certain embodiments, the formulation of the pharmaceutical compositions is parenteral administration, which may be injectable or sterile powder for injection.

In another aspect, the present invention discloses methods of the above proteins as active ingredients to treat acute pancreatitis, which comprise administering the effective dose of the above proteins to individuals.

In one embodiment, the acute pancreatitis is severe acute pancreatitis. The individuals are mammal, wherein preferably human.

In another aspect, the present invention discloses a method of establishing in vitro pancreatitis model, comprising the following steps:

a) the isolation and culture of mammalian pancreatic acinar cell;

b) add arginine with a final concentration of 2.5-10 mg/ml to cultured mammalian pancreatic acinar cells, and culture over 6 h.

### BRIEF DESCRIPTION OF THE DRAWINGS

FIG. 1: Reg 4 increases the survival rate of mice having acute pancreatitis;

FIG. 2A: the change of serum amylase;

FIG. 2B: the change of lipase;

FIG. 3: the change of pancreatic histopathology (scale=50 μm);

FIG. 4: the detection of pancreatic tissue MPO activity (*p<0.05);

FIG. 5A-D: the mRNA expression of inflammatory mediators in pancreatic tissue by semi-quantitative RT-PCR (*p<0.05);

FIG. 6: the serum levels of IL-1β by ELISA assay (*p<0.05);

FIG. 7A-B: TUNEL detects apoptosis of pancreatic cells (Bars=100 μm, *p<0.05);

FIG. 8: immunofluorescence detects the amylase of rat primary pancreatic acinar cell (×400 times);

FIG. 9: the change of lactate dehydrogenase release rate;

FIG. 10: CCK-8 detects acinar cell survival;

FIG. 11: Hoechst33342/PI double staining detects acinar cell necrosis and apoptosis (×400);

FIG. 12: TUNEL detects acinar cell apoptosis (×400 times);

FIG. 13A-B: Reg4 inhibits the acinar cell death induced by arginine (Arg);

FIG. 14: Hoechst 33342/PI double staining detects acinar cell necrosis and apoptosis.

### DETAILED DESCRIPTION

In the present article, the term of “Regenerating gene (Reg)” family belongs to the calcium-dependent phytohemagglutinin superfamily. Reg family is divided into four subtypes: Reg 1, 2, 3 and 4, wherein Reg2 is not expressed in human. Reg4 is a new Reg family member which was screened out by Hartupee [Hartupee, J. C., et al., family: Reg IV. Biochim Biophys Acta, 2001. 1518(3): p. 287-93] from a library of inflammatory bowel disease. The expression of Reg4 shows tissue specificity with high expression in gastrointestinal tract and low in normal pancreatic tissue. The Reg4 protein promotes the proliferation and growth of several tumor cells. Its expression is significantly increased in the colon, stomach, pancreatic and prostate cancer, and inflammatory bowel disease. The human Reg4 gene (NM_{—}032044.2) is 1285 bp, and CDS length is 477 bp. The front 66 bp of CDS encodes a signal peptide with a length of 22 amino acids. The signal peptide is not included in the secreted Reg4.

The preferred protein mentioned in this invention is human Reg4 and its mutants; functional active fragment or an analogue; homologues with a high degree of homology, and the vector encoding a protein containing the amino acid sequence described in SEQ ID NO. 1, such as the DNA vector (plasmid or viral). The functional active fragment or the analogue can be provided by adding, inserting, modifying, replacing or deleting one or more amino acid residues in the described amino acid sequences.

The term “analogs” includes a chimeric protein, fusion protein, anti-idiotypic antibodies, a precursor of the above compounds and other functional equivalents or mimetics, and also includes synthetic products which can simulate binding activity of Reg4.

The term “mutant” refers to the mutants of Reg4 having the amino acid sequence described in SEQ ID NO. 1. Compared to the natural Reg4 protein, the mutants enhance the activity and/or change the stereospecificity. The mutants of the natural protein may be prepared by introducing appropriate changes to the nucleotides in this invention, or by synthesize desired polypeptide in vitro. These mutants include deletion, insertion or replacement of the residues in the amino acid sequence. By a combination of deletion, insertion and replacement, the constructs are obtained, and the final protein products are generated.

The homology of the protein was defined by the GAP (Needleman and Wunsh, 1970) analysis (GCG program), wherein the gap creation penalty=5, gap extension penalty=0.3. When the minimum sequence length analyzed is 15 amino acids, the GAP analysis tests the region which is at least 15 amino acids of the two tested sequences. More preferably, when the minimum sequence length analyzed is 50 amino acids, the GAP analysis tests the region which is at least 50 amino acids of the two tested sequences. More preferably, when the minimum sequence length analyzed is 100 amino acids, the GAP analysis tests the region which is at least 100 amino acids of the two tested sequences. More preferably, when the minimum sequence length analyzed is 250 amino acids, the GAP analysis tests the region which is at least 250 amino acids of the two tested sequences. Even more preferably, when the minimum length analyzed is 500 amino acids, the GAP analysis tests the region which is in at least 500 amino acids of the two tested sequences.

The present invention also includes Reg4 protein analogues which are modified during or after synthesis. For example, the protein may be modified by biotinylation, benzylation, glycosylation, acetylation, phosphorylation, known protecting/blocking group, proteolytic cutting action, connection to antibody molecule or other cellular ligands, and so on. These modifications may be used to increase the stability and/or biological activity of Reg4 protein in this invention.

### Protein Expression

This invention includes the encoding sequences of Reg4 protein, and the vectors and transformants containing these DNA sequences.

Reg4 protein can be prepared in accordance with China patent application (Application No.: 200910056336.9) or according to the known information of human Reg4 gene (NM_{—}032044.2) by technologists in this field using current technology without creative modification.

In this invention, the term “transformant” (transformant) refers to the host cell with foreign DNA molecule.

The invention also includes a method of producing the protein by synthesis and recombinant techniques. By the known method in this field, polynucleotide (DNA or RNA), vector, transformant and organism may be isolated and purified.

The vector used in this invention may be bacteriophage, plasmid, cosmid, mini-chromosome, virus or retrovirus vectors. The polynucleotide vector that can be used for cloning and/or expressing the present invention is that it can replicate and/or express the polynucleotide in the host cell. Generally speaking, polynucleotides and/or vectors may be used for any eukaryotic or prokaryotic cells, including mammalian cells (such as human (e.g. HeLa), monkey (e.g. Cos), rabbit (e.g. rabbit reticulocytes), rat, hamster (e.g. CHO, NSO, and baby hamster kidney cells) or mouse cells (L cells)), plant cells, yeast cells, insect cells or bacterial cells (such as E. coli). Examples of vectors suitable for various types of host cells may be found in F. Ausubel et al, Current Protocols in Molecular Biology, Greene Publishing Associates and Wiley-Interscience (1992) and Sambrook et al (1989). A large number of proteins used for drugs, diagnostic reagents, vaccines and therapeutics may be expressed in the host cells containing these polynucleotides.

Variety of methods has been developed for the polynucleotide connecting with vectors via the complementary sticky ends. For example, complementary homologous sequence can be inserted into the vector DNA. The hydrogen bonding between the complementation homologous tails of the vector and DNA segment are connected to form a recombinant DNA molecule.

The synthetic linkers containing one or more restriction sites provide another method of connecting DNA segment and the carrier. The bacteriophage T4 DNA polymerase or E. coli DNA polymerase I is used to treat DNA segment generated by endonuclease restriction digestion to remove the protruding γ-single-strand end with their 3′,5′-exonuclease activity; and to blunt the 3′-recessed end with their polymerization activity. Therefore, the combination of these activities generates a DNA segment with the flat end. Then, the flat end fragments and molar excess of linker molecules is incubated at the presence of an enzyme that can catalyze ligation of blunt end DNA molecules, such as the presence of phage T4 DNA ligase. Therefore, the reaction product is the DNA segments carrying polymeric linker sequences at their ends. And then with a suitable restriction enzyme cleavage of these DNA segments, and connected to the expression vector which has been cleaved by a enzyme to produce compatible ends with the previously said DNA segment. The synthetic linkers containing multiple restriction endonuclease sites are available commercially.

The polynucleotide may be easily linked to a suitable promoter that is compatible for expression of the polynucleotide in host cells. The promoter may be a strong promoter and/or an inducible promoter. Some examples of the promoters include bacteriophage λPL promoter, the Escherichia coli lac, trp, the phoA, tac promoter, SV40 early and late promoters and retrovirus LTR promoter. Other suitable promoters are known to the technologists in the field. In addition, expression recombinant vectors contain transcription initiation and termination site, and a ribosome binding site for translation in the transcription region. The coding sequence of the recombinant vector includes the initiation codon locating at the beginning and the termination codon (UAA, UGA or UAG) locating at the end of the sequence for translation to polypeptide.

As described above, the expression vectors include at least one selection marker. The markers may be dihydrofolate reductase, G418, glutamine synthase or neomycin resistance for the eukaryotic cells; the tetracycline, kanamycin or ampicillin resistance gene for the E. coli and other bacterial cultures. Representative examples of appropriate hosts include but not limited to: bacterial cells, such as E. coli, Streptomyces and Salmonella typhimurium cells; fungal cells, such as yeast cells (such as Saccharomyces cerevisiae or Pichia pastoris); insect cells, such as Drosophila S2 and Spodoptera SF9 cells; animal cells such as CHO, COS, NSO, 293 and Bowes melanoma cells; and plant cells. The appropriate culture medium and culture conditions above are known to the field.

In order to effectively isolate, purify, or secrete the target protein, the protein may be tagged with protein or polypeptided Tag (Tag). The commonly used Tags are Glutathione-S-transferase (GST), hexahistidine peptide (His.Tag), protein A, cellulose binding domain, and so on. In the form of fusion protein the target protein obtains specific properties from the protein or polypeptide Tag. The target protein may be isolated and purified effectively due to the special nature of the protein or polypeptide Tags. For example, the His.Tag can bind to Ni-Chelating Sepharose column specifically. To obtain the target protein, the tagged protein is digested with a site-specific protease, such as thrombin, enterokinase, or factor Xa, etc. to remove the Tag sequences after purification.

This invention also includes a host cell containing the nucleotide sequence of the present invention. The nucleotide sequence is connected with one or more control area (such as promoter and/or enhancers) via well known techniques. The expression of the inserted gene can be regulated, or the host strain can be selected in accordance with the desired modification and processing of the gene product. In the presence of certain inducers, the expression under some promoters may be up-regulated. Therefore, it is possible to control the expression of the polypeptide modified genetically. Furthermore, different host cells have characteristic and special translation, post-translational processing and modification (eg, phosphorylation, cleavage) mechanisms for protein. Appropriate cell lines can be selected to ensure the needed modification and processing of the exogenous protein expressed in the host cell.

Using the method of the calcium phosphate transfection, DEAE-glucan mediated transfection, cationic lipid mediated transfection, electroporation, transduction, infection or other method, the nucleic acid and recombination vector containing the nucleic acid in this invention can be introduced into the host cell. The method is described in several standard laboratory manual, such as Davis et al., Basic Methods In Molecular Biology (1986).

The polynucleotide encoding the protein in this invention can be linked to the vector containing selection markers for replication in the host cell. Generally speaking, plasmid vector can be introduced into the host cells in sediment, such as calcium phosphate precipitate or its charged lipid compounds. If the vector is virus, the appropriate packaging cell lines can be used in vitro to package the vector, which will be used to transfect host cells.

Through the well known technology, transformed cells can be identified successfully, which contain the recombinant DNA vector in this invention. For example, the cell transduced with the recombination vector may be cultured to produce the target polypeptide. After collecting and lysis of the cells, using method described by Southern (1975) J. Mol. Biol. 95, 503 or Berent et al (1985) Biotech. 3, 208, detection of the recombinant DNA or the target protein in the supernatant by the antibody can be achieved.

It is advantageous using the well known methods to recover and purify the invention mentioned protein from cell cultures producing the recombinant protein. The methods include the ammonium sulfate or ethanol precipitation, acid extraction, anion or cation exchange chromatography, phosphocellulose chromatography, hydrophobic interaction chromatography, affinity chromatography, hydroxyapatite chromatography, hydrophobic charge action chromatography, and agglutinin chromatography. In some embodiments, it may be purified using high performance liquid chromatography (HPLC).

In some embodiments, one or more chromatography methods above may be used to purify the protein of this invention. In other embodiments, the following one or more column chromatography methods may be used to purify the fusion protein in this invention. The chromatography columns are Q-Sepharose FF column, SP Sepharose FF column, Q sepharose High Performance column, Blue Sepharose FF column, Blue column, Phenyl Sepharose FF column, DEAE Sepharose FF, a Ni-Chelating Sepharose FF column or Methyl column, and so on.

In addition, the methods described in the international publication No. WO00/44772 (the full text is incorporated herein as reference) may be used to purify the protein of this invention. Technologists in the field can easily change the methods to purify the protein of this invention. The protein of this invention can be obtained as a recombinant product produced by recombinant technology using prokaryotic or eukaryotic host cells, for example, bacterial, yeast, higher plant, insect and mammalian cells.

### Pharmaceutical Formulation

The pharmaceutical formulations in the present invention or the formulations containing the proteins are described in the present invention. The proteins could be mixed with one or more pharmaceutically acceptable carriers or excipients to meet the needs of various administration methods, such as tablets, capsules, powders, granules, syrup, solution, oral liquid, spiritus agents, tinctures, aerosols, powders, injections, sterile powder for injection, suppository, etc.

“Pharmaceutically acceptable” components are suitable for human beings and/or animals, and without excessive adverse side effects (such as toxicity, irritation and allergic reaction). It means that the benefit/risk factor is reasonable. “Pharmaceutically acceptable carriers” are the acceptable solvents, suspending agents or excipients used in pharmacy or foods for the purpose of delivery of the proteins of the present invention to the animals or human beings. The carriers could be liquids or solids.

The proteins of the present invention could be administered via the routes like oral, intravenous, intramuscular or subcutaneous.

The above formulations administered orally may be tablets, capsules, powders, granules, syrups, solutions, spiritus agent. The solid state carriers include starch, lactose, dicalcium phosphate, microcrystalline cellulose, sucrose, kaolin, micronized silica, talc, low-substituted hydroxypropyl cellulose, sodium carboxymethyl starch, polyvinyl pyrrolidone. The liquid state carriers include sterile water, ethanol, polyethylene glycol, non-ionic surfactants and edible oils (such as corn oil, peanut oil and sesame oil). Adjuvants normally used in the process of preparing a pharmaceutical composition are: flavoring agents, coloring agents, preservatives (such as hydroxyalkyl phenylalkyl-butyl ester, sodium benzoate, sorbic acid), and anti-oxidants (such as vitamin E, vitamin C, naphthalene sodium sulfite and dibutylhydroxytoluene).

The formulations for parenteral administration include injection and sterile lyophilized powder. Both are mixtures of drugs and one or several pharmaceutically acceptable carriers or excipients for parenteral administration. The solvents include sterile water, ethanol, glycerol, propylene glycol and polyethylene glycol. Besides, bacteriostats (e.g., benzyl alcohol, butyl paraben, thiomersalate), isoosmotic adjustment agents (e.g., sodium chloride, glucose), suspending agents (e.g., sodium carboxymethylcellulose, methylcellulose), solubilizers (e.g., Tween-80, phosphatidylcholine), antioxidants (e.g., Vitamin E, Vitamin C, sodium pyrosulfite) and stuffing bulking agents (e.g., maltose, mannitol) are needed.

From the standpoint of easy preparation and administration, the preferred pharmaceutical formulation is a solid state formulation, especially in the lyophilized powder for injection.

The pharmaceutical formulation of the invention can be prepared according to the well known and recognized methods meeting the pharmaceutical production requirements. The pharmaceutical composition contains the invention mentioned protein, the pharmaceutically acceptable carrier, and required unit dose. The pharmaceutical composition of the invention may contain a prodrug form of the invention mentioned protein. The prodrug in the recipient host body can be metabolic conversion to the active form of the invention mentioned protein. The pharmaceutical composition of the invention can also be combined with other therapies, such as simultaneous, sequential or separate application. The pharmaceutical composition of the invention can include other active substrates.

### APPLICATIONS OF THE INVENTION

The invention mentioned protein is effective in the treatment of acute pancreatitis, it may provide new options for the clinical treatment of acute pancreatitis in the future.

We disclose the method using the invention protein as an active ingredient to prevent or treat acute pancreatitis. The method comprises administering an effective dose of the above (a) or (b) mentioned protein to patients.

The “effective dose” or the “therapeutic amount” is intended to mean an amount sufficient to produce a therapeutic effect. The effective amount can be divided into one or multiple administration. Typically, an effective amount is sufficient to alleviate, improve, stabilize, slow down or delay the further development of the disease.

The effective dose of the active ingredient used can vary with the mode of administration and the severity of the disease to be treated. For most large mammals, the daily total dose is about 0.01-1000 mg of the active ingredient. Normally, the adult clinical dose range of 0.01-200 mg/day, preferably 0.05-100 mg/day.

The following text further illustrates the invention with the specific embodiments. It should be understood that these embodiments are merely used to illustrate the invention and not for limiting the scope of the invention. The experimental methods of the following implementation which have no specific condition are usually done under conditions in accordance with the conventional conditions or the conditions recommended by the manufacturer.

Technologists in the field can prepare the Reg4 protein in accordance with China patent application (Application No.: 200910056336.9) or according to the known human Reg4 gene (NM_{—}032044.2) information using prior technology without creative change of the methods.

### Example 1

### Reg4 Protein Attenuate the Severity of Pancreatitis In Vivo

### 1. Materials and Methods

### 1.1 Materials

### 1.1.1 Experimental Animals

C57BL/6 male mice (SPF grade, 25-30 g, 8 W-10 W) were purchased from SLACCAS, Shanghai, China, and maintained in air-filtered units at 23 for one week before the experiment. Food and water were provided freely to them before the experiment.

### 1.1.2 Main Reagents:

DANase I: TAKARA company; proteinase K: the TAKARA Company; MPO activity in high-quality sensitive colorimetric quantitative detection kit: GENMED company; In Situ Cell Death Detection Kit POD: Roche company; Mouse IL-1/IL-1F2 Immunoassay: R & D company.

### 1.2 Methods

### 1.2.1 Murine Experimental Groups:

In order to observe the changes in the mortality of acute pancreatitis in mice, C57BL/6 mice were randomly divided into NS group (n=40) and Reg4 group (n=40, 300 μg Reg4/kg sc q12 h), 4.5 g/kg L-Arg-HCl intraperitoneally injected twice with 1 h intervals. Mortality rates of the two groups of mice were observed within 7 days.

Pharmacology experiment: C57BL/6 mice were randomly divided into three groups, the control group (n=12), the NS group (saline group, n=36) and Reg4 group (n=36, 300 μg Reg4/kg, sc. q12 h). All mice were intraperitoneally injected with 4 g/kg L-Arg-HCl twice with interval of 1 h. The control group was injected with an equal volume of saline. The time is set to 0 hour after the second injection of L-Arg-HCl. Mice were sacrificed at day 2, 3 and 4, respectively. 12 mice for the Reg4 group, 4 mice for the control group were sacrificed at each indicated time point.

### 1.2.2 Serum Amylase and Lipase Detection

Fully automatic biochemical analyzer was used to measure serum amylase and lipase levels.

### 1.2.3 Pancreatic Tissue Myeloperoxidase (MPO) Activity Detection

Pancreatic MPO activity was measured using MPO activity and quality sensitive colorimetric quantitative detection kit (GENMED) according to the instructions.

Activity calculation: [(sample reading−background reading)×0.25 (reaction system, ml)×sample dilution]÷[0.01 (Sample volume, ml)×30 (milli molar light absorbtion coefficient)×0.6 (cm)×30 (reaction time, minutes)]=units/ml÷(diluted sample protein concentration) mg/ml=units/mg×1000=units/g. Unit=micromoles of chloramine/minute.

### 1.2.4 Pancreatic Histopathological Score

Pancreatic tissues are fixed in 4% paraformaldehyde, and conventional dehydration, embedding, sectioning, staining were performed. Pancreatic tissue semi-quantitative scoring is referred to the literature [Toma, H., et al., Gastroenterology, 2000, 119 (5): p. 1373-81], and Table 1.

TABLE 1
Histopathologic Grading Scale for Pancreatitis

| | score |
|---|---|
| Edema | |
| No edema | 0 |
| Focal expansion of interlobar septa | 0.5 |
| Diffuse expansion of interlobar septa | 1 |
| Same as 1 + focal expansion of interlobular septa | 1.5 |
| Same as 1 + diffuse expansion of interlobular septa | 2 |
| Same as 2 + focal expansion of interacinar septa | 2.5 |
| Same as 2 + diffuse expansion of interacinar septa | 3 |
| Same as 3 + focal expansion of intercellular spaces | 3.5 |
| Same as 3 + diffuse expansion of intercellular spaces | 4 |
| Parenchymal necrosis | |
| No necrosis | 0 |
| Focal occurrence of 1-4 necrotic cells/HPF | 0.5 |
| Diffuse occurrence of 1-4 necrotic cells/HPF | 1 |
| Same as 1 + focal occurrence of 5-10 necrotic cells/HPF | 1.5 |
| Diffuse occurrence of 5-10 necrotic cells/HPF | 2 |
| Same as 2 + focal occurrence of 11-16 necrotic cells/HPF | 2.5 |
| Diffuse occurrence of 11-16 necrotic cells/HPF | 3 |
| Same as 3 + focal occurrence of >16 necrotic cells/HPF | 3.5 |
| >16 Necrotic cells/HPF | 4 |
| Inflammation and perivascular infiltrate | |
| No inflammation | 0 |
| 2-5 Leukocytes/HPF | 0.5 |
| 6-10 Leukocytes/HPF | 1 |
| 11-15 Leukocytes/HPF | 1.5 |
| 16-20 Leukocytes/HPF | 2 |
| 21-25 Leukocytes/HPF | 2.5 |
| 26-30 Leukocytes/HPF | 3 |
| >30 Leukocytes/HPF or focal microabscesses | 3.5 |
| >35 Leukocytes/HPF or confluent microabscesses | 4 |
| Vacuolization | |
| No vacuolization | 0 |
| Less than 1/8 of cells had vacuolization | 0.5 |
| Between 1/8 and 2/8 of cells had vacuolization | 1 |
| Between 2/8 and 3/8 of cells had vacuolization | 1.5 |
| Between 3/8 and 4/8 of cells had vacuolization | 2 |
| Between 4/8 and 5/8 of cells had vacuolization | 2.5 |
| Between 5/8 and 6/8 of cells had vacuolization | 3 |
| Between 6/8 and 7/8 of cells had vacuolization | 3.5 |
| All cells had vacuolization | 4 |

HPF, high-power field.

1.2.5 Semi-Quantitative RT-PCR Analysis the Levels of IL-1β, IL-6 and TNF-α mRNA in Pancreatic Tissue.

(1) extraction of total RNA from pancreatic tissue;

(2) Synthesis the first-strand cDNA by reverse transcriptase using 2 μg total RNA;

(3) PCR primer sequences: As shown in Table 2, all primers were synthesized by Shanghai Shangon. Ltd.

(4) PCR reaction.

TABLE 2
Primer Sequence Table

| Primers | Forward (5′ → 3′) | Reverse (5′ → 3′) |
|---|---|---|
| IL-1β | TCATGGGATGATGATGATAACCTGCT | CCCATACTTTAGGAAGACACGGAT |
| IL-6 | CCGGAGAGGAGACTTCACAG | GGAAATTGGGGTAGGAAGGA |
| TNF-α | AGTCCGGGCAGGTCTACTTT | AAGCAAAAGAGGAGGCAACA |
| β-actin | GTCCACCTTCCAGCAGATGT | AGGGAGACCAAAGCCTTCAT |

1.2.6 ELISA assay of serum IL-1β

The serum IL-1β was analyzed by ELISA method using Mouse IL-1/IL-1F2 Immunoassay kit (R&D Company) according to the manufacturer's protocols.

### 1.2.7 Quantification of Apoptotic Cells

The numbers of apoptotic cells (cell nuclear stained with green fluorescent) were counted in randomly chosen 10 fields under 200 time magnification for each section.

### 1.2.8 Statistical Analysis

Quantitative data are expressed as mean±SEM. Statistical analysis was performed using SAS 8.0 software package. The survival curves were compared using the log-rank test. Two groups were compared using the Manne-Whitney non-parametric U test. P value<0.05 was considered to be statistically significant.

### 2. Results

### 2.1 Reg4 Protein Reduced the Mortality of Arginine Induced Mice Acute Pancreatitis

35 arginine-induced acute pancreatitis mice (control group) were dead during 7 days, 5 mice survived, and the mortality rate is 87.5%. Comparably, only 22 Reg4-treated mice were dead during 7 days, 18 mice survived, and the mortality rate is 55%. Only one Reg4-treated mouse was dead during first 24 hours, meanwhile, 7 mice in the control group of the arginine-induced acute pancreatitis mice were dead. The difference of survival curves between the two group mice is statistically significant (FIG. 1, p<0.01).

### 2.2 Reg4 Protein Reduced the Severity of Arginine-Induced Mice Acute Pancreatitis

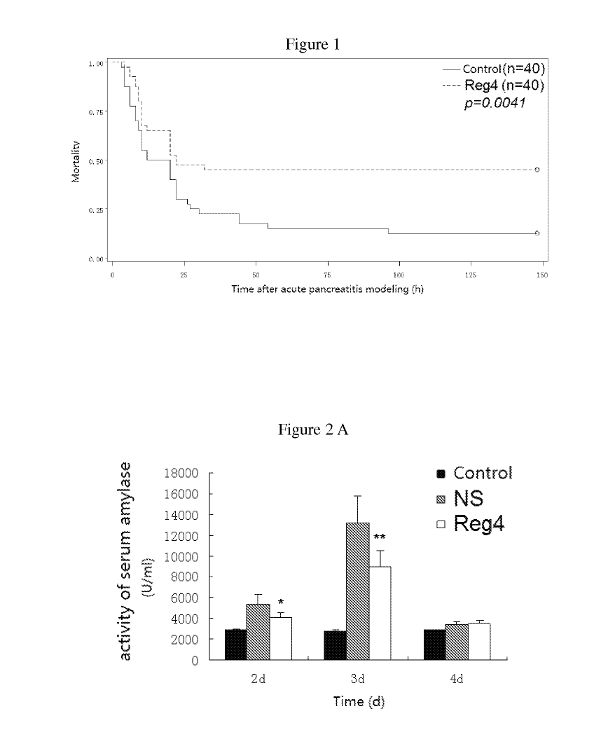

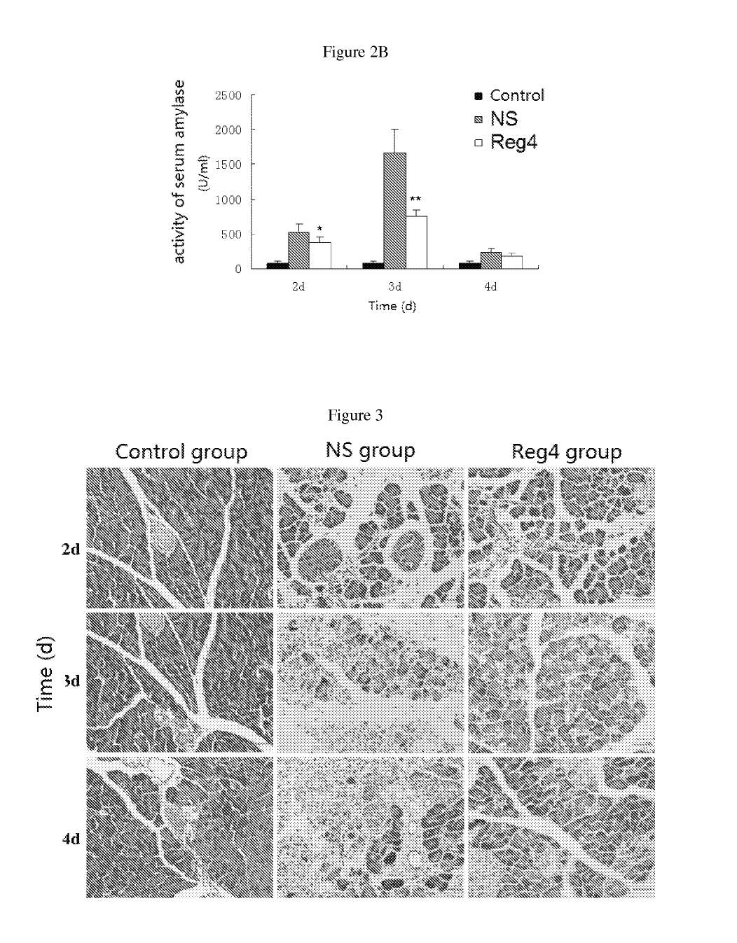

The levels of amylase in the serum of arginine-induced acute pancreatitis mice (saline group) were 5399±876, 13168±2604, 3473±286 (U/ml); while the Reg4 treatment group were 44101±479, 8990±1526, 3502±317 (U/ml), at day 2, 3 and 4, respectively. The levels of lipase in the serum of saline group were 528±120, 1663±341, 244±52 (U/ml); while the Reg4 treatment group were 382±78, 758±96, 181±48 (U/ml), at day 2, 3 and 4, respectively. Markedly decreased amylase and lipase activities were observed in the sera of Reg4-treated mice at day 2 and 3 compared with that in the saline group (amylase, p<0.05; lipase, p<0.01), as shown in FIGS. 2A and 2B.

Histopathology examination of the pancreas showed pancreatic interstitial edema, vacuolization of acinar cells, focal and foliated necrosis, and many inflammatory cells infiltration in the saline group as shown in FIG. 3. The severity of acinar cells necrosis and inflammatory cells infiltration were significantly reduced in the Reg4 treatment group, especially obvious on day 3 and 4. Using histopathologic Grading Scale (Table 1) to determine the severity of pancreatitis, the histopathologic score in the Reg4 treatment group was significantly lower than that of the normal saline group, and the score data are 6.8±1.5, 9.9±1.8, 8.5±1.9 in the saline group vs 5.4±1.4, 7.4±1.9, 5.5±1.0 in the Reg4 group at day 2, 3 and 4, respectively (p<0.05).

### 2.3 Reg4 Protein Reduced MPO Activity in Pancreatic Tissues of Arginine-Induced Mice Acute Pancreatitis

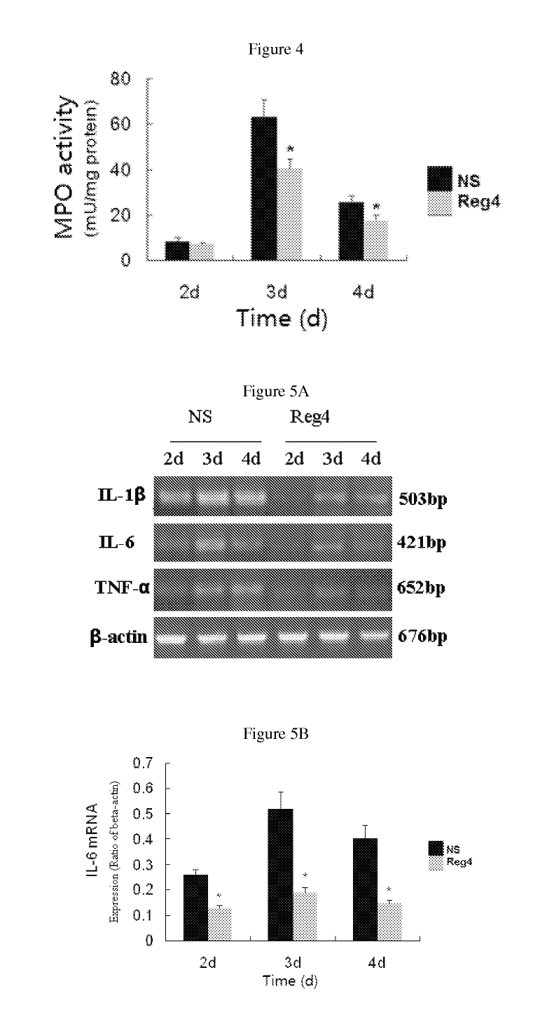

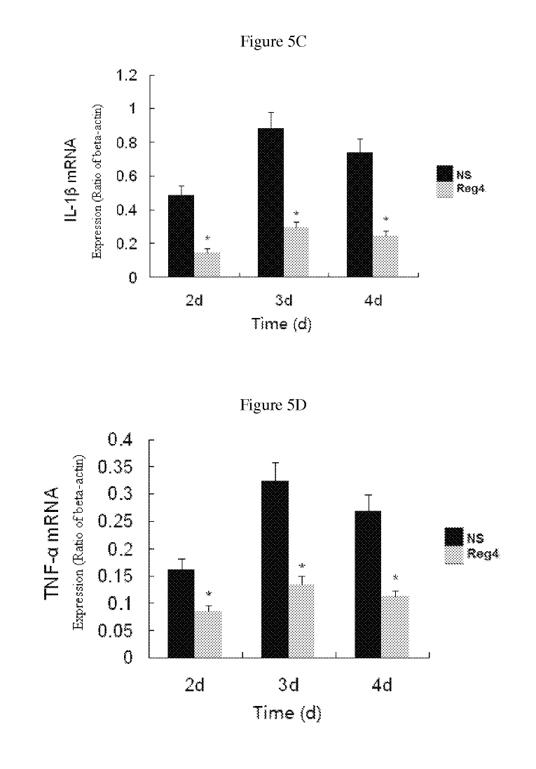

MPO exists in the polymorphonuclear leukocytes particularly the neutrophils and monocytes' azurophilic granules. Thus, its activity reflects the degree of infiltration of neutrophils to tissues. The MPO activities in the mice pancreatic tissues of saline group are 8.4±1.7, 63.3±7.6, 25.4±3.0 (activity units/g protein) at day 2, 3 and 4, respectively. Correspondingly, the Reg4 treatment group are 7.0±0.9, 40.4±4.0, 17.6±2.3 (activity units/g protein). The data at day 3 and 4 of the Reg4 treatment group are significantly lower than the saline group (p<0.05), as shown in FIG. 4.

### 2.4 Reg4 Protein Inhibited the Expression of Inflammatory Genes

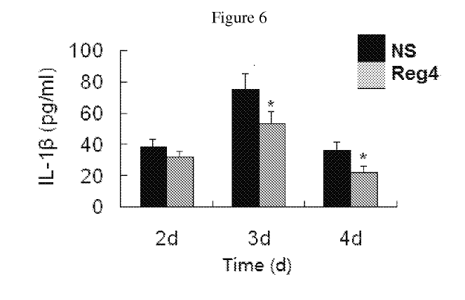

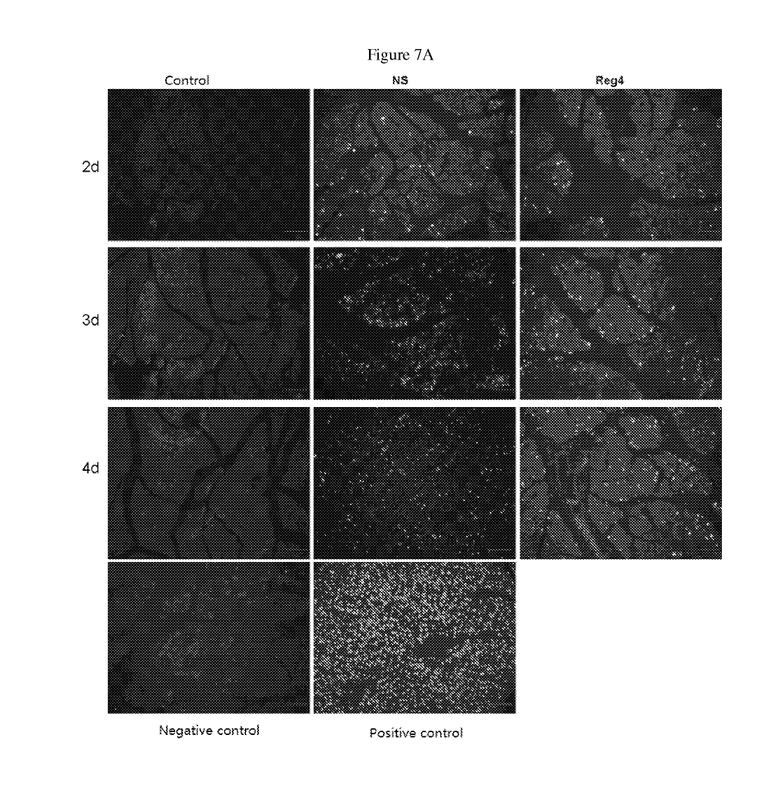

Electrophoresis of RT-PCR products is shown in FIG. 5A-D: in the saline group, IL-1β, IL-6 and TNF-α mRNA level reached its maximum at days 3, and then come down. Compared with the saline group, the IL-1β, IL-6 and TNF-α mRNA level in the Reg4 treatment group are significantly decreased. We further examined the serum IL-1β levels. Serum IL-1β in the Reg4 treatment group is significantly lower than that in the saline group, especially obvious on day 3 and 4 (the data were 39±4.0, 75±10.0, 36±5.0 vs 32±3.5, 53±7.4, 22±4.2, pg/ml), as shown in FIG. 6.

### 2.5 Reg4 Protein Inhibited Arginine-Induced Pancreatic Acinar Cell Death

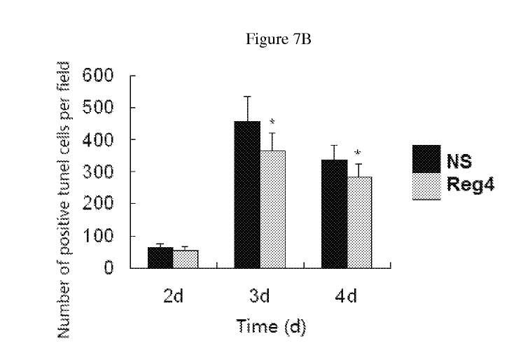

The degree of acinar cell death is positively proportional to the severity of acute pancreatitis. The death of acinar cell includes two forms of necrosis and apoptosis. Acinar cell necrosis in the Reg4 treatment group was significantly reduced compared with the saline group, as shown in FIG. 3. There was not only acinar cell necrosis, but also acinar cell apoptosis in the Arginine-induced acute pancreatitis as shown by TUNEL assay. Acinar cell apoptosis in the Reg4 treatment group was also slightly reduced than the saline group, especially on day 3 and 4, as shown in FIG. 7A/B.

The present study found that, Reg4 protein significantly reduced the mortality of arginine induced mice acute pancreatitis. 24 hours after high-dose arginine administration, urea, alanine aminotransferase enzyme (ALT) and aspartate aminotransferase (AST) were significantly increased, accompanied by acidosis. The pH value dropped from 7.56±0.38 to 5.81±0.81. And these may be some of the reasons for the death of experimental animals within 24 hours. We found that Reg4 protein reduced mice death mostly after 24 hours, which strongly suggest that Reg4 protein mainly reduced mice death caused by arginine-induced acute pancreatitis. Acinar cell necrosis and inflammatory responses play important role in the development of acute pancreatitis. Acinar cell necrosis is an important source for inflammatory response, while the production and release of inflammatory factors increase cell necrosis. The interaction between acinar cell necrosis and inflammatory response creates a vicious cycle, and jointly promotes the development of acute pancreatitis. Pancreatic histopathology examination revealed Reg4 protein significantly reduced the acinar cell necrosis and inflammation in mice pancreatitis. Reg4 protein also significantly reduced MPO, IL-1β, IL-6 and TNF-α mRNA levels in pancreatic tissue and IL-1β level in serum, which suggest that Reg4 protein may have anti-inflammatory and anti-necrotic properties.

In acute pancreatitis, acinar cell death includes two forms of necrosis and apoptosis [Kaiser, A M, et al, Am J Physiol, 1995, 269 (5 Pt 1): p. C1295-304; Gukovskaya, A S, et al., Gastroenterology, 1996. 110 (3): p. 875-84.]. Necrosis/apoptosis ratio is positively correlated with the severity of experimental pancreatitis. [Gukovskaya, A S and S J Pandol, General Surgery, 2004. 4 (6): p. 567-86]. The conventional view is that induction of acinar cells apoptosis could reduce pancreatitis injury, for apoptotic cells could be phagocytized by macrophages around which will not produce the inflammatory response. However, Miwa at al. [Miwa, K., et al, Nat Med, 1998, 4 (11): page 1287-92] reported that inoculation of the tumor cells expressing Fas ligand to wild type mice could cause severe leukocyte infiltration, which is absent in the IL-1α/β gene knockout mice. The data suggest that apoptosis could also induce inflammation and challenge the traditional view of the apoptosis does not provoke inflammation. Additionally, acinar cell apoptosis plays an important role in chronic pancreatitis for acinar cell atrophy [Bateman, A. C., et al., Gut, 2002. 50(4): p. 542-8]. Therefore, the role of apoptosis in pancreatic repair and regeneration after acute pancreatitis need to be further studied.

The present study also found that, in arginine-induced mice acute pancreatitis models, acinar cell death includes two forms of necrosis and apoptosis, and the necrosis is the mainly form. Reg4 protein not only significantly inhibits arginine-induced acinar cell necrosis, but also partially inhibits acinar cell apoptosis though not significantly. Recent studies have shown that necrosis includes two forms of accident and programmed necrosis. Programmed necrosis and apoptosis may have common signaling pathway [Proskuryakov, S. Y, A. G Konoplyannikov, and V. L. Gabai, Exp Cell Res, 2003. 283(1): p. 1-16; Edinger, A. L. and C. B. Thompson, Curr Opin Cell Biol, 2004. 16(6): p. 663-9]. For example, FasL and TNF-α not only induces cell apoptosis, but also induces cell necrosis when apoptosis is suppressed [Vercammen, D., et al., Cytokine, 1997, 9 (11): p. 801-8; Karunanayake, E H, D J Hearse, and G Mellows, Biochem Soc Trans, 1975. 3 (3): p. 410-4.]. Therefore, we hypothesized that in the arginine induced acinar cell death, apoptosis signaling pathway may be suppressed, which lead the cells to necrosis and expressed as acute necrotizing pancreatitis. Reg4 protein may inhibit the common pathway of necrosis and apoptosis, thereby inhibits the arginine induced acinar cell necrosis and apoptosis.

In summary, Reg4 protein inhibits necrosis and inflammatory reaction of pancreatic acinar cells, thereby reduces the mortality and severity of experimental pancreatitis.

### Example 2

### Reg4 Inhibits the Death of Pancreatic Acinar Cells In Vitro

### 1. Materials and Methods

### 1.1 Materials

Penicillin/streptomycin, DMEM/Ham F-12 medium, Fetal bovine serum, Trypsin/EDTA are all from Gbico Co.; BSA, collagenase I, PI are all from Sigma; 200 mesh stainless steel sieve cells is from BD Biosiences; Trypsin inhibitor, Aprotinin are from Ameresco; Cell Counting Kit-8 is from Dojindo; Trypan blue is from Invitrogen; Hoechest 33342 and RIPA lysis buffer are from Beyotime; RevertAid Premium First Strand cDNA Synthesis Kit is from Fermentas; PrimeScript™ RT reagent Kit is from TAKARA; PVDF membrane is from Millipore; mouse β-actin monoclonal antibody, mouse amylase monoclonal antibody, horseradish peroxidase-conjugated goat anti-rabbit IgG secondary antibody, horseradish peroxidase-conjugated goat anti-mouse IgG secondary antibody are all from Santa Cruz; rabbit BCL-2 monoclonal antibody, rabbit BCL-xl monoclonal antibody are from Cell signaling; Donkey anti-mouse IgG is from Jackson ImmunoResearch; ECL is from Pierce Co.

### 1.2 Methods

### 1.2.1 Isolation and Culture of Rat Pancreatic Acinar Cells

1) Isolation and Culture of Acinar Cells:

The improved collagenase digestion method is used.

(1) 4 week-old SD rat (about 100 g, Shanghai Laboratory Animal Center, Shanghai, China) were fast for 12 h, but drinks. They were anesthetized by intraperitoneal injection (3% sodium pentobarbital). After bleeding they were sterilized by immersing in 75% alcohol 5 minutes. The rats were fixed in the tissue culture hood, opened peritoneally, looked for duodenum and pancreas, and pancreas were quickly took out which is about 1 g (1 cm×1 cm).

(2) Pancreas was cut under sterile condition, rinsed with PBS (containing 0.01% Trypsin inhibitor) first. Interstitial tissue membrane and excess tissue were removed, and then the pancreas was cut into 1 mm³ fragments.

(3) The pancreas fragments were moved into 10 mL of the preheated (37° C.) isolation solution I (0.02% Trypsin, 0.25% EDTA), and shaked for 5 minutes in 37° C. water bath.

(4) Centrifuge at 500 rpm for 2 minutes, discard supernatant.

(5) Rinse cells with culture medium, centrifuge at 500 rpm for 2 minutes, discard supernatant.

(6) Add 20 mL digestion solution II (0.1 mg/mL collagenase I, 0.25 mg/mL collagenase IV, 20% FCS, 5% BSA, 0.1 mg/mL Trypsin inhibitor, 0.01 mg/mL Aprotinin), incubate at 37° C. for 45 minutes rotating at 120-140 round/min

(7) The cell suspension was filtered through sterile stainless steel mesh (pore size 200 mesh).

(8) Collect cell suspension, count, centrifuge at 1000 rpm for 2 minutes, wash 1 to 2 times with cell culture medium.

(9) Plate the cells into 96-well culture plate at 1×10⁴ per well, incubate at 37° C. in a humidified CO₂ incubator overnight, and then replace with fresh culture medium, use for subsequent experiments.

2) Cell Treatment and Groups

(1) The acinar cells were plated into 96-well culture plate at 1×10⁴ per well. 0, 2.5, 5, 10 mg/mL L-arginine were add to the cell cultures respectively. The cells were incubated for 6 h, 12 h and 24 h, respectively, Cell viability was detected by trypan blue exclusion and CCK-8.

(2) Acinar cells were plated into 96-well culture plate at 1×10⁴ per well, or into 6-well culture plate at 1×10⁵ per well 5 mg/mL L-arginine was added. At the same time different concentrations of Reg4 protein (0, 4, 8, 16, 32 μg/ml) was added, and the cells were cultured for 12 h. Cell viability was detected by trypan blue exclusion, LDH release rate and CCK-8. Total RNA and protein were extracted from the cell collections.

1.2.2 Cell smear and HE Staining:

Cell suspension diluted appropriately was made. The cells were spinned to the glass slide using the slide spin machine. The slides were fixed at room temperature for 20 minutes in 4% paraformaldehyde, and stained with routine HE method.

### 1.2.3 Immunofluorescence Staining:

After the cell smears were fixed, the expression of amylase was detected by immunofluorescence staining.

### 1.2.4 Trypan Blue Exclusion Assay:

It is used to assess the necrosis of acinar cells.

The single cell suspension was prepared, and diluted appropriately (1×10⁶/ml).
1. Staining: 90 μl cell suspension was transferred into 1.5 mL tube, 10 μl 0.4% trypan blue was added and mixed.
2. Count: The live and dead cells were counted using Hemocytometer (the dead cells are stained light blue, while the living cells have no staining).
3. Calculation of the living cell rate (%)=the total number of living cells/(the total number of living cells+the total number of dead cells)×100%

### 1.2.5 Lactate Dehydrogenase (LDH) Release Rate:

It is used to assess the damage of the acinar cells.

The acinar cells were incubated with arginine and/or Reg4 for the appropriate time, and the culture supernatant was collected. Lysis buffer was added to the cell culture and the lysate was collected. LDH level was measured in the supernatant and lysate by conventional biochemical colorimetric.

LDH release rate (%)=LDH in supernatant/(LDH in lysates+LDH in supernatant)×100%.

### 1.2.6 Detection of Percentage of Live Cells by CCK-8.

The acinar cells were incubated with arginine and/or Reg4 for the appropriate time. 10 μL per well CCK-8 solution was added, and incubated at 37 for 1 h. OD450 nm was measured. Simultaneously blank wells (culture medium, CCK-8 solution), control wells (cells, the buffer for Reg4 protein, culture medium, CCK-8 solution), were set at 6 wells per group.

Calculate the percentage of live cell=(the Reg4 treated cells−control)/(control−blank)×100%.

### 1.2.7 Detection of Apoptosis and Necrosis by Hoechst 33342/PI Double Staining.

Both PI and Hoechst 33342 can hind nuclear DNA or RNA. But PI can not penetrate through the normal cell membrane. Hoechst 33342 as a fluorescent dye is membrane permeable. The cell membrane is damaged in the necrotic or late stage apoptotic cells, which could be stained red by PI. Normal cells and early apoptotic cells could be stained by Hoechst. The normal nucleus stained by Hoechst is round, light blue with darker blue particles; while the nucleus of apoptotic cells are stained bright blue due to condensation, or nucleus may appear as lobulated, fragmented, and locate to the peripheral of the cells. Thus, the PI stained cells are necrotic cells; Apoptotic cells are stained with Hoechst as bright blue, or lobulated nucleus, and peripheral localized nucleus. There are four kinds of cell morphologies by Hoechst/PI double staining at UV excitation under fluorescence microscope:

Live cells: stained blue, nuclei shows normal structure.

Early apoptotic cells: stained blue, nuclei is condensed or bead-like.

Late apoptotic cells: stained red, but nuclei shows condensed chromatin.

Non-apoptotic death cells: stained red, nuclei shows normal structure.

The acinar cells were incubated with arginine and/or Reg4 protein for the appropriate time. 10 μg/ml Hoechst 33342 was added and the cells were cultured for 15 minutes. After centrifugation, washed once with PBS, the cells were resuspended in pre-cooled PBS. 50 μg/ml PI was added at 4° C. for 1 minute before observation, photograph and counting under fluorescence microscopy.

### 1.2.8 Detection of Apoptosis by TUNEL Assay

The cells were collected and the cell suspension was prepared. Cell smear in glass slides was prepared, and fixed in acetone for 5 minutes. TUNEL staining was performed as guided by the commercial reagent kit.

### 1.2.9 Statistical Analysis

Quantitative data are presented as mean±SEM. Statistical analysis is performed using SAS 8.0 sofeware. The two groups are compared by non-parametric Mann-Whitney U test. Multiple groups are examined by one-way ANOVA. P<0.05 is accepted as significant.

### 2. Results

### 2.1 Culture and Identification of Rat Pancreatic Acinar Cells

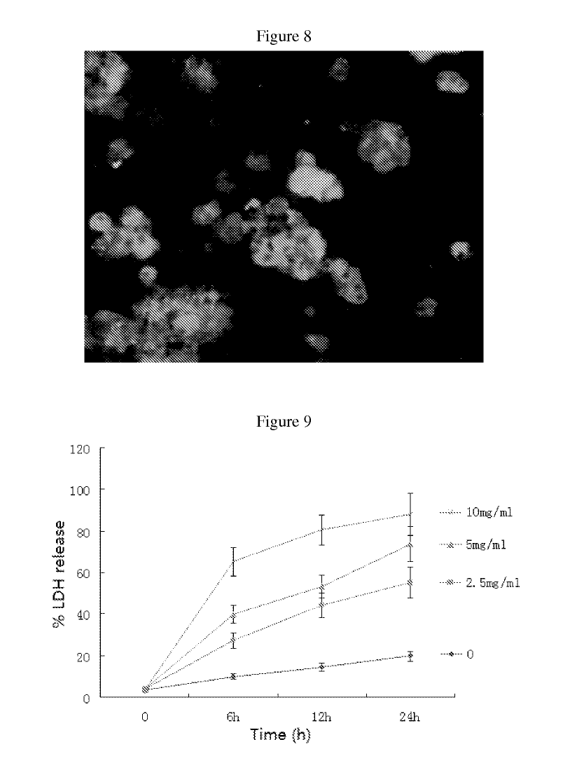

The pancreatic acinar cells did not adhere to culture plate for growth under phase contrast microscope. They appeared as cell clusters gathered into groups with clear cell boundary, strong light reflection, and rich in zymogen. The expression of amylase in the primary cultured cells was detected by cellular immunofluorescence method. The results show that almost all cultured cells expressed amylase. Thereby these cells are determined as the pancreatic acinar cells as shown in FIG. 8.

### 2.2 Arginine-Induced Death of Pancreatic Acinar Cells

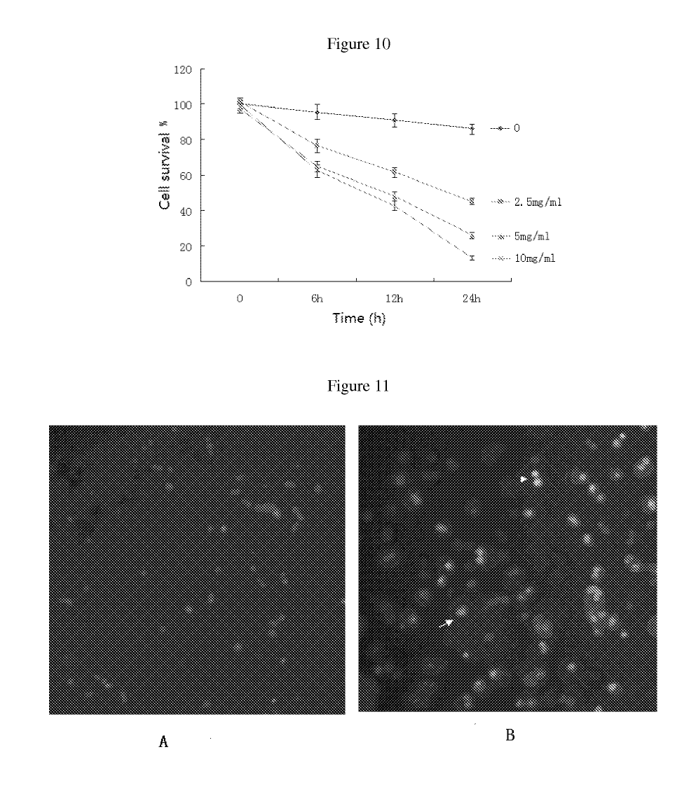

By trypan blue staining, the cell viability of freshly isolated rat pancreatic acinar cells was high, more than 95%. With the extension of culture time in vitro, the cell viability decreased to some extent, the cell viability was more than 80% at 24 h after culture. The pancreatic acinar cells were incubated with different concentration of arginine. The cell viability decreased significantly in a time and dose dependent fashion. Cell viability was less than 15% at 24 h in 10 mg/ml arginine group, as shown in Table 3. Meanwhile, as shown in FIG. 9, when acinar cells were incubated with 2.5-10 mg/ml arginine for 6 h, LDH release rate increased significantly in a dose and time dependent fashion. When the ainar cells were treated with different concentrations of arginine for 6 h, 12 h and 24 h, the results of cell viability by CCK-8 showed decline of acinar cell viability in a time and dose dependent fashion, as shown in FIG. 10.

TABLE 3
Change of rat pancreatic cell viability (%, Mean ± SEM)

| Incubation | Arginine | | | |
|---|---|---|---|---|
| time | 0 mg/ml | 2.5 mg/mL | 5 mg/mL | 10 mg/mL |
| 0 h | 95.5 ± 3.8 | 94.9 ± 2.8 | 96.2 ± 4.3 | 95.7 ± 3.8 |
| 6 h | 90.7 ± 5.1 | 78.4 ± 4.9* | 61.4 ± 3.9* | 57.1 ± 5.2** |
| 12 h | 85.4 ± 3.9 | 63.8 ± 3.4* | 45.8 ± 2.6** | 40.3 ± 4.1** |
| 24 h | 81.3 ± 4.3 | 45.9 ± 2.8** | 23.9 ± 4.4** | 12.7 ± 2.5** |

Note:
compared with the control group,
*P < 0.05,
**P < 0.01.

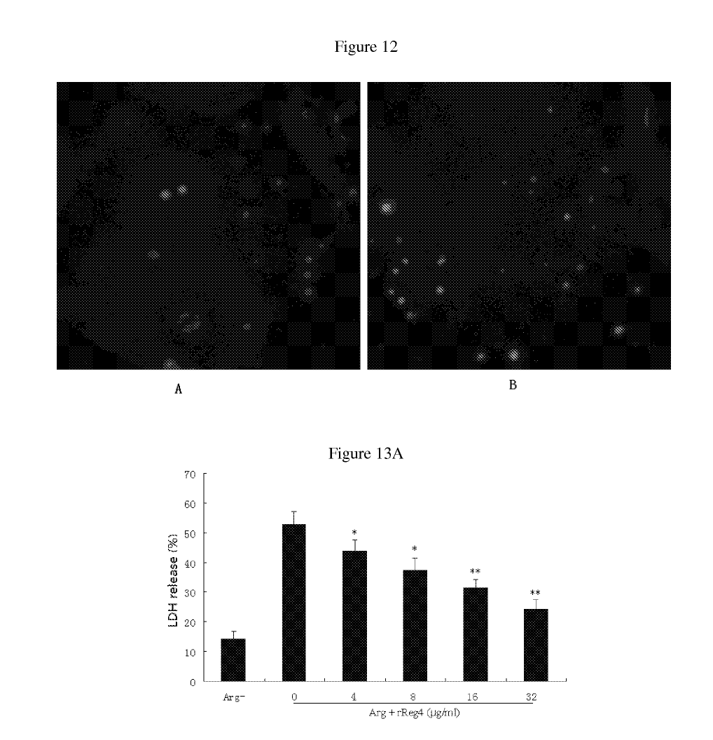

The detection of acinar necrosis and apoptosis by PI/Hoechst33342 double staining showed that arginine induced the death of acinar cell mostly in the form of necrosis, much less in the form of apoptosis. Nucleus stained red are necrotic cells, while nucleus stained dark blue are apoptotic cells, as shown in FIG. 11 (A. control group, B. arginine treated group). Very few apoptotic acinar cells induced by arginine were detected by TUNEL assay, as shown in FIG. 12 (A. control group, B. arginine treated group).

### 2.3 Reg4 Protein Inhibited Arginine-Induced Death of Acinar Cells

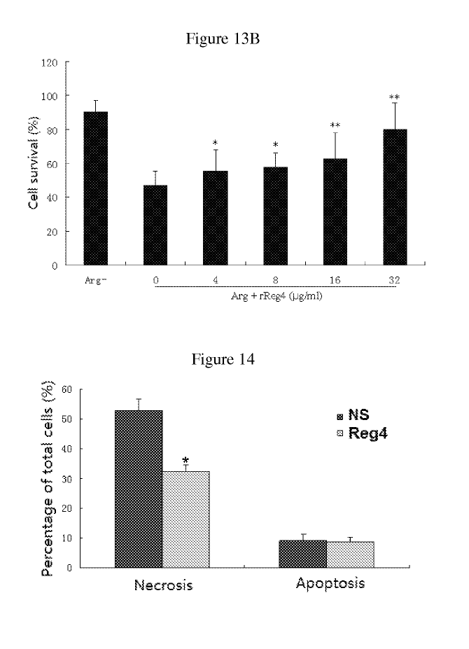

Compared with the arginine group (Arg+0 μg/ml rReg4), the lactic dehydrogenase release in the Reg4 group (Arg+rReg4 at 4, 8, 16, and 32 μg/ml) is significantly lowered at each concentration (4, 8 μg/ml, p<0.05; 16, 32 μg/ml, p<0.01), as shown in FIG. 13A. On the other hand, the CCK-8 assay showed Reg4 at 4-32 μg/ml significantly increased the survival rate of acinar cell (4, 8 μg/ml, p<0.05; 16, 32 μg/ml, p<0.01) as shown in FIG. 13B. PI/Hoechst33342 double-staining showed that the necrosis of acinar cells is 52.8±3.8% and apoptosis is 9.8±1.9% after 12 h incubation with 5 mg/ml arginine. The necrosis and apoptosis are 32.5±2.1% and 8.6±1.5% respectively in the presence of Reg4 μl 6 μg/ml. Compared with the control group, the necrosis is significantly lower (p<0.05), and the apoptosis is slightly lower than the control group, but without statistical significance (FIG. 14).

There is no report about the effect of arginine treatment on primary acinar cells. Our study presents that arginine induces injury of pancreas acinar cells directly in vitro. The injury is primarily necrosis and less apoptosis, and it is related to the concentration of arginin and treatment time. Treating acinar cells with 5 mg/ml arginin for 12 h primarily cause necrosis, which is similar to the type of injury in acute pancreatitis in vivo. Thus, it is an ideal pancreatitis model in vitro. We also found that Reg4 protein could inhibit arginine induced acinar cell necrosis in vitro.

### EQUIVALENTS

The scope of this invention is not restricted by the particular embodiments described in this application, which are intended as single illustrations of individual aspects of the invention. The scope of this invention also includes the methods and components with the same function of Reg4. Many modifications and variations of this invention can be made without departing from its spirit and scope, as will be apparent to those skilled in the art. Such modifications and variations are intended to fall within the scope of the appended claims. The full texts of the literatures cited are included in this application for reference.

What is claimed is:

1. A protein described as follows in (a) or (b) that has application for preparing drugs treating acute pancretitis:
(a) a protein whose amino acid sequence is shown as SEQ ID NO. 1;
(b) a protein whose amino acid sequence has at least 70% homology compared with the amino acid sequence described in (a) and has activity for treating acute pancretitis.

2. According to the application described in claim 1, the feature of the described acute pancreatitis is acute severe pancreatitis.

3. According to the application described in claim 1, the feature of the described protein sequence (b) is as shown in SEQ ID NO. 2.

4. A pharmaceutical composition comprising the following components and given weight percentage:
The protein described in claim 1 comprises 1%˜99%;
Pharmaceutically acceptable drug carrier or excipient comprises the remaining amount of the composition.

5. A pharmaceutical composition described in claim 4, the feature of formulation is parenteral drug.

6. A pharmaceutical composition described in claim 5, the feature of the formulation is injection or sterile freeze-dried powder for injection.

7. A method of treating acute pancreatitis, the feature includes giving an effective dose of the protein described in claim 1 to an individual.

8. According to the method described in claim 7, the feature of the described acute pancreatitis is acute severe pancreatitis.

9. According to the method described in claim 7, the feature of the described individual is mammal.

10. According to the method described in claim 9, the feature of the described mammal is human.

11. A method of establishment of an in vitro pancreatitis model, including following steps:
a) Isolation and primary culture of mammalian pancreas acinar cells;
b) Addition of arginine to the final concentration of 2.5˜10 mg/ml to the primary cultured mammalian pancreas acinar cells for at least 6 h;
c) Detection of the survival rate of the acinar cells.

12. According to the method described in claim 11, the feature of described mammal is rat.

13. According to the method described in claim 11, the feature of the described Step a includes the following steps:
1) 4 week-old SD rat, fasting for 12 h with free drinking, are anesthetized with intraperitoneal injection 3% pentobarbital. The rats were sacrificed by bleeding, and sterilized by sinking in 75% alcohol. Pancreas is cut off in aseptic condition, washed in PBS including 0.01% Trypsin inhibitor. After removing the interstitial tissue membrane, the pancreas is cut into 1 mm³ pieces;
2) Tissues are transferred into 37° C. preheated isolation solution containing 0.02% trypsin and 0.25% EDTA, and digested at 37° C. for 5 min;
3) Centrifugation at 500 rpm for 2 min, remove supernatant;
4) Tissues are washed by culture medium and harvested by 500 rpm for 2 min;
5) Tissues are further digested in solution containing 0.1 mg/ml collagenase I, 0.25 mg/ml collagenase IV, 20% FCS, 5% BSA, 0.1 mg/ml trypsin, 0.01 mg/ml trasylol at 37° C. for 45 min;
6) The digested cell suspension is filtered by 200 mesh stainless strainer;
7) The cell suspension is collected and counted, and centrifuged by 1000 rpm. The cells are further washed 1-2 times with culture medium;
8) The cell concentration is adjusted to 10⁵/cm², plated into 96-well plate with 10⁴/well, and cultured in 37° C., 5% CO₂ overnight. Fresh culture medium is changed before next experiment.

14. According to the method described in claim 11, the feature of the described Step b is treating the acinar cells with final concentration of 5 mg/ml arginine for 12 h.

15. According to the method described in claim 11, the feature of the described Step c is using trypan blue exclusion assay, LDH release rate, or CCK-8 methods to detect cell survival rate.
